(19) United States
(12) Patent Application Publication
(10) Pub. No.: US 2024/0029864 A1
(43) Pub. Date: Jan. 25, 2024

(54) META-LEARNING OF PATHOLOGIES FROM RADIOLOGY REPORTS USING VARIANCE-AWARE PROTOTYPICAL NETWORKS

(71) Applicant: Covera Health, New York, NY (US)

(72) Inventors: Arijit Sehanobish, Washington, DC (US); Kawshik Kannan, Jersey City, NJ (US); Nabila Abraham, Rochester Hills, MI (US); Anasuya Das, Teaneck, NJ (US); Benjamin Odry, West New York, NJ (US)

(21) Appl. No.: 18/226,008

(22) Filed: Jul. 25, 2023

Related U.S. Application Data
(60) Provisional application No. 63/392,033, filed on Jul. 25, 2022.

(51) Int. Cl. G16H 30/20; G06N 20/00

(52) U.S. Cl. CPC G16H 30/20; G06N 20/00

(57) ABSTRACT

A process can include performing meta-learning for a variance-aware prototypical network pre-trained on a dataset comprising examples of a first type of radiology report associated with a single domain. The meta-learning comprises learning one or more prototype representations for each radiology classification task and a variance information for the prototype representations of each radiology classification task. The one or more respective prototype representations for each radiology classification task are modeled as a Gaussian and a query sample comprising text data of a type of radiology report seen during the meta-learning is provided to the variance-aware prototypical network. A distance metric is determined between a Dirac distribution representation of the query sample and the Gaussians of the respective prototype representations for each radiology classification task included in the meta-learning. The query sample is classified based on identifying a respective prototype representation having the smallest distance metric.

### CROSS-REFERENCE TO RELATED APPLICATIONS

This application claims the benefit of priority to U.S. Provisional Patent Application No. 63/392,033, filed Jul. 25, 2022, and entitled “META-LEARNING OF PATHOLOGIES FROM RADIOLOGY REPORTS USING VARIANCE-AWARE PROTOTYPICAL NETWORKS,” which is hereby incorporated by reference, in its entirety and for all purposes.

### TECHNICAL FIELD

The present disclosure relates generally to automated pathology detection in medical imaging, and more specifically pertains to a systems and techniques for automated pathology detection using prototypical networks (e.g., ProtoNets).

### BACKGROUND

Various machine learning models can be used to perform tasks such as Natural Language Processing (NLP). For example, the use of large, pre-trained Transformer-based language models such as BERT (Bidirectional Encoder Representations from Transformers) and GPT (Generative Pre-trained Transformer) have changed the machine learning-based NLP landscape. However, fine tuning such models to perform more specific tasks (and/or domain-specific tasks) often requires a large quantity of training examples for each target task that the model is being trained to perform. As such, annotating multiple datasets and training these models on various downstream tasks can quickly become time consuming and expensive to perform.

Few-shot learning (FSL) is a machine learning (ML) approach that allows models to be trained with small amounts of labeled data. FSL can be used to provide a neural network (e.g., a neural network classifier, etc.) with improved generalization to new tasks containing only a few samples with supervised information. For example, an FSL-based neural network classifier may attempt to correctly classify one or more classes that are previously unseen (e.g., unseen during training) but are known based on a set of labeled support samples (e.g., provided during inference). In some cases, FSL-based neural network classifiers can classify a given query (e.g., inference input) into one or more of a closed set of pre-defined classes that were seen in training, or into a previously unseen class that is identified during an FSL episode (e.g., based on the support samples).

There is a need for new systems and techniques that can be used to perform meta-learning for NLP tasks. For example, there is a need for meta-learning algorithms for NLP that can be used to apply meta-learning to a pre-trained transformer-based machine learning network (e.g., BERT) for various downstream text classification tasks.

### BRIEF DESCRIPTION OF THE DRAWINGS

The accompanying drawings are presented to aid in the description of various aspects of the disclosure and are provided solely for illustration of the aspects and not limitation thereof.

FIG. 1 illustrates an example implementation of a System-on-a-Chip (SoC), in accordance with some examples;

FIG. 2A and FIG. 2B illustrate an example of a fully connected neural network, in accordance with some examples;

FIG. 3 is a diagram illustrating an example of a few-shot learning (FSL) scenario, in accordance with some examples;

FIG. 4 is a diagram illustrating an example architecture and workflow that can be used to implement a variance-aware prototypical machine learning network, in accordance with some examples;

FIG. 5 is a diagram illustrating an example segmentation output corresponding to a cervical spine medical imaging dataset, in accordance with some examples;

FIG. 6 is a diagram illustrating an example segmentation output corresponding to a knee medical imaging dataset, in accordance with some examples; and

FIG. 7 illustrates an example computing system that can be used to implement various aspects described herein.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. Additional features and advantages of the disclosure will be set forth in the description which follows, and in part will be obvious from the description, or can be learned by practice of the herein disclosed principles. It will be appreciated that for simplicity and clarity of illustration, where appropriate, reference numerals have been repeated among the different figures to indicate corresponding or analogous elements. The description is not to be considered as limiting the scope of the embodiments described herein.

### Overview

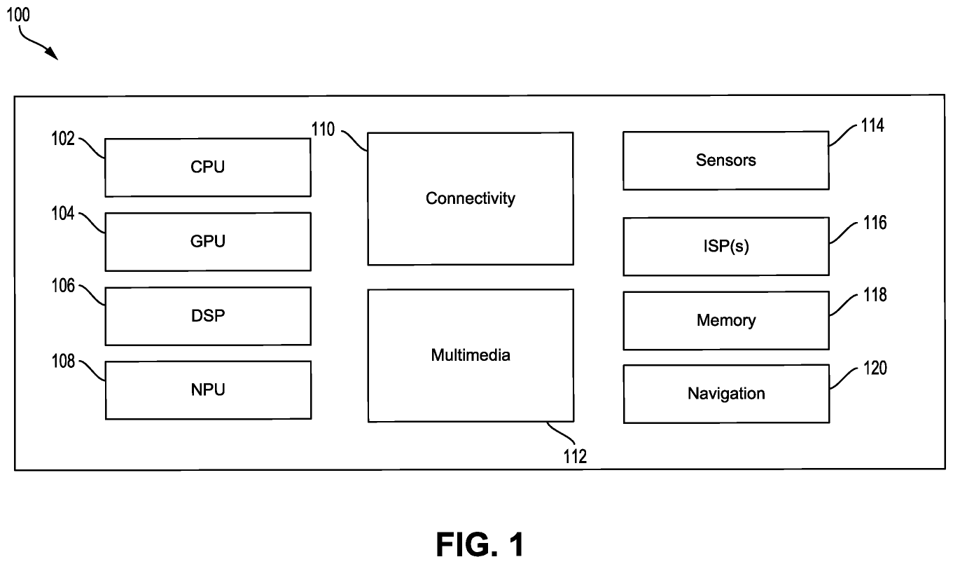

FIG. 1 illustrates an example implementation of a system-on-a-chip (SoC) 100, which may include a central processing unit (CPU) 102 or a multi-core CPU, configured to perform one or more of the functions described herein. Parameters or variables (e.g., neural signals and synaptic weights), system parameters associated with a computational device (e.g., neural network with weights), delays, frequency bin information, task information, among other information may be stored in a memory block associated with a neural processing unit (NPU) 108, in a memory block associated with a CPU 102, in a memory block associated with a graphics processing unit (GPU) 104, in a memory block associated with a digital signal processor (DSP) 106, in a memory block 118, and/or may be distributed across multiple blocks. Instructions executed at the CPU 102 may be loaded from a program memory associated with the CPU 102 or may be loaded from a memory block 118. The SoC 100 may also include additional processing blocks tailored to specific functions, such as a GPU 104, a DSP 106, a connectivity block 110, which may include fifth generation (5G) connectivity, fourth generation long term evolution (4G LTE) connectivity, Wi-Fi connectivity, USB connectivity, Bluetooth connectivity, and the like, and a multimedia processor 112 that may, for example, detect and recognize gestures, speech, and/or other interactive user action(s) or input(s). In one implementation, the NPU is implemented in the CPU 102, DSP 106, and/or GPU 104. The SoC 100 may also include a sensor processor 114, image signal processors (ISPs) 116, and/or navigation module 120, which may include a global positioning system. In some examples, the sensor processor 114 can be associated with or connected to one or more sensors for providing sensor input(s) to sensor processor 114. For example, the one or more sensors and the sensor processor 114 can be provided in, coupled to, or otherwise associated with a same computing device. The SoC 100 may be based on an ARM instruction set. In an aspect of the present disclosure, the instructions loaded into the CPU 102 may comprise code to search for a stored multiplication result in a lookup table (LUT) corresponding to a multiplication product of an input value and a filter weight. The instructions loaded into the CPU 102 may also comprise code to disable a multiplier during a multiplication operation of the multiplication product when a lookup table hit of the multiplication product is detected. In addition, the instructions loaded into the CPU 102 may comprise code to store a computed multiplication product of the input value and the filter weight when a lookup table miss of the multiplication product is detected.

Machine learning (ML) can be considered a subset of artificial intelligence (AI). ML systems can include algorithms and statistical models that computer systems can use to perform various tasks by relying on patterns and inference, without the use of explicit instructions. One example of a ML system is a neural network (also referred to as an artificial neural network), which may include an interconnected group of artificial neurons (e.g., neuron models). Neural networks may be used for various applications and/or devices, such as speech analysis, audio signal analysis, image and/or video coding, image analysis and/or computer vision applications, Internet Protocol (IP) cameras, Internet of Things (IoT) devices, autonomous vehicles, service robots, among others.

Individual nodes in a neural network may emulate biological neurons by taking input data and performing simple operations on the data. The results of the simple operations performed on the input data are selectively passed on to other neurons. Weight values are associated with each vector and node in the network, and these values constrain how input data is related to output data. For example, the input data of each node may be multiplied by a corresponding weight value, and the products may be summed. The sum of the products may be adjusted by an optional bias, and an activation function may be applied to the result, yielding the node's output signal or “output activation” (sometimes referred to as a feature map or an activation map). The weight values may initially be determined by an iterative flow of training data through the network (e.g., weight values are established during a training phase in which the network learns how to identify particular classes by their typical input data characteristics).

Different types of neural networks exist, such as convolutional neural networks (CNNs), recurrent neural networks (RNNs), generative adversarial networks (GANs), multilayer perceptron (MLP) neural networks, transformer neural networks, among others. For instance, convolutional neural networks (CNNs) are a type of feed-forward artificial neural network. Convolutional neural networks may include collections of artificial neurons that each have a receptive field (e.g., a spatially localized region of an input space) and that collectively tile an input space. RNNs work on the principle of saving the output of a layer and feeding this output back to the input to help in predicting an outcome of the layer. A GAN is a form of generative neural network that can learn patterns in input data so that the neural network model can generate new synthetic outputs that reasonably could have been from the original dataset. A GAN can include two neural networks that operate together, including a generative neural network that generates a synthesized output and a discriminative neural network that evaluates the output for authenticity. In MLP neural networks, data may be fed into an input layer, and one or more hidden layers provide levels of abstraction to the data. Predictions may then be made on an output layer based on the abstracted data.

Deep learning (DL) is one example of a machine learning technique and can be considered a subset of ML. Many DL approaches are based on a neural network, such as an RNN or a CNN, and utilize multiple layers. The use of multiple layers in deep neural networks can permit progressively higher-level features to be extracted from a given input of raw data. For example, the output of a first layer of artificial neurons becomes an input to a second layer of artificial neurons, the output of a second layer of artificial neurons becomes an input to a third layer of artificial neurons, and so on. Layers that are located between the input and output of the overall deep neural network are often referred to as hidden layers. The hidden layers learn (e.g., are trained) to transform an intermediate input from a preceding layer into a slightly more abstract and composite representation that can be provided to a subsequent layer, until a final or desired representation is obtained as the final output of the deep neural network.

As noted above, a neural network is an example of a machine learning system, and can include an input layer, one or more hidden layers, and an output layer. Data is provided from input nodes of the input layer, processing is performed by hidden nodes of the one or more hidden layers, and an output is produced through output nodes of the output layer. Deep learning networks typically include multiple hidden layers. Each layer of the neural network can include feature maps or activation maps that can include artificial neurons (or nodes). A feature map can include a filter, a kernel, or the like. The nodes can include one or more weights used to indicate an importance of the nodes of one or more of the layers. In some cases, a deep learning network can have a series of many hidden layers, with early layers being used to determine simple and low-level characteristics of an input, and later layers building up a hierarchy of more complex and abstract characteristics.

A deep learning architecture may learn a hierarchy of features. If presented with visual data, for example, the first layer may learn to recognize relatively simple features, such as edges, in the input stream. In another example, if presented with auditory data, the first layer may learn to recognize spectral power in specific frequencies. The second layer, taking the output of the first layer as input, may learn to recognize combinations of features, such as simple shapes for visual data or combinations of sounds for auditory data. For instance, higher layers may learn to represent complex shapes in visual data or words in auditory data. Still higher layers may learn to recognize common visual objects or spoken phrases. Deep learning architectures may perform especially well when applied to problems that have a natural hierarchical structure. For example, the classification of motorized vehicles may benefit from first learning to recognize wheels, windshields, and other features. These features may be combined at higher layers in different ways to recognize cars, trucks, and airplanes. Neural networks may be designed with a variety of connectivity patterns. In feed-forward networks, information is passed from lower to higher layers, with each neuron in a given layer communicating to neurons in higher layers. A hierarchical representation may be built up in successive layers of a feed-forward network, as described above. Neural networks may also have recurrent or feedback (also called top-down) connections. In a recurrent connection, the output from a neuron in a given layer may be communicated to another neuron in the same layer. A recurrent architecture may be helpful in recognizing patterns that span more than one of the input data chunks that are delivered to the neural network in a sequence. A connection from a neuron in a given layer to a neuron in a lower layer is called a feedback (or top-down) connection. A network with many feedback connections may be helpful when the recognition of a high-level concept may aid in discriminating the particular low-level features of an input.

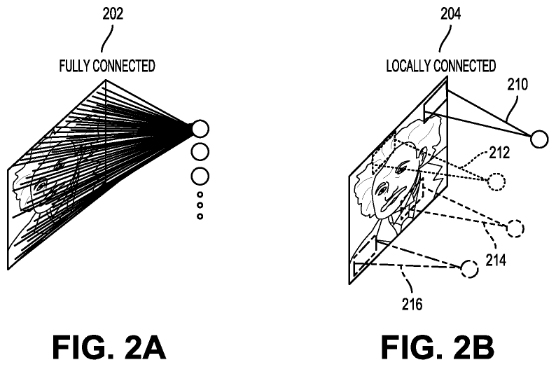

The connections between layers of a neural network may be fully connected or locally connected. FIG. 2A illustrates an example of a fully connected neural network 202. In a fully connected neural network 202, a neuron in a first layer may communicate its output to every neuron in a second layer, so that each neuron in the second layer will receive input from every neuron in the first layer. FIG. 2B illustrates an example of a locally connected neural network 204. In a locally connected neural network 204, a neuron in a first layer may be connected to a limited number of neurons in the second layer. More generally, a locally connected layer of the locally connected neural network 204 may be configured so that each neuron in a layer will have the same or a similar connectivity pattern, but with connections strengths that may have different values (e.g., 210, 212, 214, and 216). The locally connected connectivity pattern may give rise to spatially distinct receptive fields in a higher layer, as the higher layer neurons in a given region may receive inputs that are tuned through training to the properties of a restricted portion of the total input to the network.

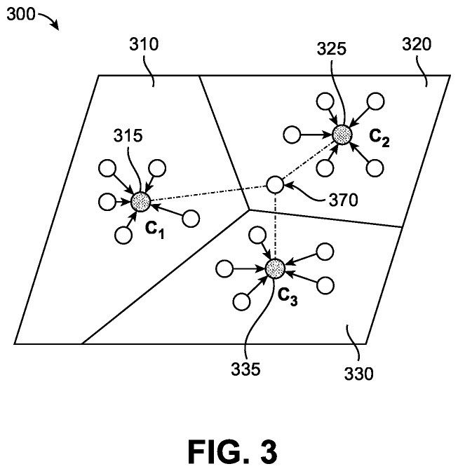

FIG. 3 is a diagram illustrating an example of a few-shot learning (FSL) scenario 300 that can be used and/or implemented in the context of one or more machine learning networks or models described herein. For instance, in some cases, a prototypical machine learning network (e.g., the presently disclosed variance-aware prototypical machine learning network) can be implemented using an FSL-based approach. FSL and FSL-based approaches can be used to perform few-shot classification, wherein a classifier (e.g., a neural network or other machine learning classifier) can generalize and extend inference to include new classes not seen in the training set, given only a small number of examples of each new class.

For example, given only a small number of support examples for each new class, an FSL-based neural network classifier can use an attention mechanism over a learned embedding of the labeled set of support examples (e.g., the support set) to predict classes for unlabeled points (e.g., a query set). In a process of query-by-example, a trained FSL-based neural network classifier can receive a support set that includes M support examples for each of N unseen classes, and a query set that includes one or more query samples. The neural network classifier can determine a prototype representation for each unseen class N (e.g., using the M support examples associated with each unseen class N). Each unlabeled query sample can be classified into one of the previously unseen classes N based on a computed distance between the query sample and each prototype representation. In some cases, this inference process can be referred to as an N-way M-shot episode, where the goal of the FSL-based neural network classifier is to correctly classify a query set into N classes that are unseen during training but known using the M support samples.

As illustrated, FIG. 3 depicts an example of a 3-way 5-shot FSL scenario, in which a neural network classifier (not shown) receives as input a support set that includes a total of 15 labeled support examples, divided into three previously unseen classes (e.g., 310, 320, and 330) having five support examples each. In one illustrative example, the 3-way 5-shot FSL scenario 300 can be associated with a prototypical network, which learns a metric space in which classification can be performed by computing distances to prototype representations of each class.

The use of prototypical networks for FSL can be based on the idea that there exists an embedding in which points cluster around a single prototype representation for each class. As illustrated in FIG. 3, the five support examples included in class 310 can be seen to cluster around prototype representation 315; the five support examples included in class 320 can be seen to cluster around prototype representation 325; the five support examples included in class 330 can be seen to cluster around prototype representation 335; etc. A prototypical network can include one or more neural networks that learn (e.g., during training) a non-linear mapping of the input into an embedding space. Using the learned embedding space, the prototypical network can take each previously unseen class's prototype to be the mean of its support set in the embedding space. For example, the prototype representation 315 can be determined as the mean of the five embedded support examples for class 310; the prototype representation 325 can be determined as the mean of the five embedded support examples for class 320; the prototype representation 335 can be determined as the mean for the five embedded support examples for class 330; etc.

Classification can then be performed for an embedded query point by determining the nearest class prototype to the query point. For example, the embedded query point 370 can be classified into class 320 based on a determination that the distance from embedded query point 370 to prototype representation 325 is smaller than the distance from embedded query point 370 to either of the remaining prototype representations 315 and 335. In some examples, embedded query points (e.g., embedded query point 370) may be classified based on the Euclidean distance between the embedded query point and each of the prototype representations, although it is noted that various other distance metrics and/or determinations may also be utilized without departing from the scope of the present disclosure.

### Example Embodiments

Described herein are systems and techniques for performing few-shot text classification using one or more prototypical machine learning networks. In one illustrative example, the systems and techniques described herein can be used to implement a variance-aware prototypical network that incorporates variance (e.g., second moment) information of the conditional distribution(s) associated with the prototypical network. A prototypical machine learning network can also be referred to interchangeably herein as a “ProtoNet” or “prototypical network.”

As will be described in greater depth below, class prototypes (e.g., as used to perform few-shot learning (FSL) and/or as utilized in a prototypical network) of the ProtoNet can be replaced with one or more Gaussians. For instance, the prototypical network conditional distribution(s) can be represented and/or modeled using one or more corresponding Gaussian representations. In some embodiments, one or more regularization terms can be used to improve the clustering of examples (e.g., queries to the variance-aware prototypical network) near an appropriate or most similar class prototype. For example, experimental results indicate that the systems and techniques described herein can be seen to outperform various strong baselines on over 13 public datasets. In some aspects, the Gaussians for each class distribution can be used to detect potential out-of-distribution (OOD) data points during deployment.

Various aspects of the present disclosure will be described with respect to the figures.

As noted previously, pre-trained Transformer-based language models (PLMs) have been achieved significant success to date in performing many NLP tasks. However, existing PLM implementations are typically trained and/or implemented based on using a large number of in-domain and labeled examples to perform finetuning (e.g., in order for the PLM implementations to successfully perform the specific NLP tasks, the PLM must first be trained or finetuned using the large number of in-domain labeled examples—“in-domain” refers to examples that are specifically tailored or suited to the context or anticipated use case of the specific NLP task).

There is a desire for PLM and other machine learning-based implementations that can be used to perform domain-specific NLP tasks without the requirement of finetuning on large volumes of in-domain labeled examples, which are time consuming and expensive to produce, obtain, maintain, etc. The general problem can also be referred to as “learning to learn,” and more specifically, learning to learn from limited supervision. Learning to learn from limited supervision is an important problem with widespread application in various technical fields and areas where obtaining labeled training data suitable for training large models may be difficult and/or expensive

As such, meta-learning methods have been proposed as effective solutions for few-shot learning (FSL). Existing applications of such meta-learning methods may provide improved performance in few-shot learning for vision tasks, such as learning to classify new image classes within a similar dataset (e.g., where an FSL-based machine learning network learns to classify new image classes that were unseen during training, based on similar classes that were seen during training). For example, on classical few-shot image classification benchmarks, the training tasks are sampled from a “single” larger dataset (e.g., Omniglot, miniImageNet, etc.), and the label space contains the same task structure for all tasks.

There has been a similar trend of such classical methods in NLP as well. However, in text classification tasks (e.g., such as NLP), the set of source tasks available during training, and the set of target tasks during evaluation, can range from sentiment analysis to grammatical acceptability judgment. Recent works have used a range of different source tasks (e.g., different not only in terms of input domain, but also in terms of task structure (e.g., label semantics, and number of labels)) for meta-training and have shown successful performance on a wide range of downstream tasks. However, meta-training on various source tasks remains challenging as it requires resistance to over-fitting to certain source tasks due to its few-shot nature and more task-specific adaptation due to the distinct nature among tasks. In some aspects, the use of meta-training for NLP machine learning implementations, rather than the use large in-domain training datasets for finetuning a PLM, can be seen to trade one training challenge for another (e.g., the challenge of obtaining and labeling the large in-domain dataset for PLM finetuning approaches vs. the challenge of implementing task-specific adaptation while avoiding over-fitting for meta-learning approaches).

In medical NLP tasks and implementations (such as those contemplated herein), collecting large number of diverse labeled datasets is difficult. For example, a data collection process can include collecting high quality labeled radiology reports and using the labeled reports to train internal annotators who then annotate unlabeled data, where the internal annotators can be humans providing manual annotations and/or can be separate ML models providing model-assisted annotations, etc. In either approach, this training process for the internal annotators can be expensive and time consuming.

There are three common approaches to meta-learning: metric-based, model-based, and optimization-based. Model agnostic meta-learning (MAML) is an optimization-based approach to meta-learning which is agnostic to the model architecture and task specification. Over the years, various variants of the method have shown that it is an ideal candidate for learning to learn from diverse tasks. However, to solve a new task, MAML type methods would require training a new classification layer for the task. In contrast, metric-based approaches, such as prototypical networks, being non-parametric in nature can handle varied number of classes and thus can be easily deployed. Prototypical networks usually construct a class prototype (mean) using the support vectors to describe the class and given a query example assigns the class whose class prototype is closest to the query vector.

For instance, as described above with respect to FIG. 3, a class prototype can be conceptually understood based on the idea that there exists an embedding in which points (when represented or encoded in the same embedding space) will cluster around a single prototype representation for each class. In many conventional implementations of prototypical networks, a prototype for a previously unseen class can be calculated as the mean (e.g., average) embedding of the respective embeddings of each support set example of the previously unseen class.

Existing approaches to prototypical networks perform classification of an embedded query point based on determining the nearest class prototype to the query point, where the distance is calculated in the shared embedding space used to represent both the class prototypes and the query point. The embedded query points (e.g., embedded query point 370 of FIG. 3) are classified based on determining the Euclidean distance between the embedded query point and each respective one of the class prototype representations.

There is a need for systems and techniques that can utilize a large, labeled dataset consisting of numerous classes to meta-train a model that can subsequently be used on a large number of downstream datasets having little to no training examples. Depending on use cases, such a model can be deployed in production and/or be used to pseudo-label data in an active learning loop to cut down on the annotation process. This is a highly non-trivial problem since the reports can be differently structured for different body parts and there can be a substantial variation in writing style across radiologists from different institutions.

As disclosed herein, a novel loss function is developed that extends existing prototypical networks. In some embodiments, a regularization term is introduced that achieves tight clustering of query examples near the class prototypes. As described in the context of the examples herein, meta-training of models may be performed on a large, labeled dataset of shoulder MRI reports (e.g., single domain) and can be seen to demonstrate good performance on four diverse downstream classification tasks on radiology reports on knee, cervical spine and chest. Superior performance of the presently disclosed systems and techniques is shown for 13 public benchmarks over well-known methods (e.g., such as Leopard). The systems and techniques described herein can be simple to train and easy to deploy (unlike gradient-based methods). In some embodiments, the systems and techniques described herein can be deployed and subsequent dataset statistics used to inform out-of-distribution (OOD) cases.

In particular, in at least some embodiments, the systems and techniques described herein can be used to implement a variance-aware prototypical network based at least in part on replacing a distance function d (e.g., as provided in Eq. (2), below) used to implement a prototypical network with a Wasserstein distance calculation, which is a true metric. For instance, in one illustrative example, the systems and techniques can implement a variance-aware prototypical network using Wasserstein distance calculations rather than the conventional use of a Euclidean distance value.

In another illustrative example, the variance-aware prototypical network(s) described herein can utilize an additional regularization term that is added to encourage the L2 norm of the covariance matrices to be small, thereby encouraging the class examples to be clustered close to the centroid. In some aspects, the systems and techniques can utilize Gaussians to represent the underlying conditional distributions of a prototypical network implementations, where the use of Gaussians is based at least in part on the explicit closed form formula of the Wasserstein distance.

Described below are example datasets that can be used to train or otherwise implement the variance-aware prototypical networks described herein. It is noted that these datasets are described for purposes of illustration only, and are not intended to be construed as limiting—various other datasets can be utilized as training datasets without departing from the scope of the present disclosure.

In one illustrative example, training datasets are MRI radiology reports detailing various pathologies in different body parts. Models are meta-trained on a dataset of shoulder pathologies which is collected from 74 unique and de-identified institutions in the United States. 60 labels are chosen for training and 20 novel labels are chosen for validation. This diverse dataset has a rich label space detailing multiple anatomical structures in the shoulder, granular pathologies associated with the anatomical structure(s), and a respective severity level for the granular pathologies in each structure. The relationship between the granularity/severity of these pathologies at different structures can be leveraged for other pathologies in different body parts and may lead to successful transfer to various downstream tasks, as will be described in greater detail below (e.g., with respect to the variance-aware prototypical networks and/or FSL-based approaches described herein).

For instance, continuing in the example above relating to the example shoulder dataset, the corresponding label space used to meta-train the machine learning models described herein can include (in one illustrative example) 80 labels. The shoulder dataset labels can correspond to factors and information such as clinical history, metadata, impressions, findings to various granular pathologies at different structures in the shoulder (e.g., AC joint, rotator cuff, muscles, bursal fluid, supraspinatus, infraspinatus, subscapularis, labrum, glenohumeral Joint, humeral head, acromial morphology, impingement: AC joint, etc.)). The labels can be split or otherwise divided across a training data subset and a validation data subset of the larger dataset, such that all pathologies in a given structure will appear in (e.g., be included in) either the training data subset or validation data subset, but do not appear in both the training and validation data subsets. In some aspects, this split of dataset labels across training and validation subsets can help the machine learning model to better learn various keywords that may describe the granularity of a pathology in a given anatomical structure of interest.

In some embodiments, meta-learning can be performed based on applying a meta-learner to a plurality of different downstream classification tasks that span different domains. For instance, continuing in the context of medical NLP and/or the baseline shoulder MRI-based dataset described above, in one illustrative example a meta-learner can be applied to four downstream medical anatomy/pathology classification tasks that span different sub-specialties (e.g., cancer screening, musculoskeletal radiology, and neuroradiology) and are both common as well as clinically important.

Each task is a downstream classification task based on the input radiology report. For instance, a lung nodule cancer screening task can be performed to correspond to a high risk cancer screening for lung nodules (According to Fleishner criterion), where the lung nodule cancer screening task buckets patients into a binary risk-based classification for lung cancer: a ‘Red’ classification indicative of a patient at high-risk of lung cancer and requiring follow-up imaging immediately (or within the next three months), and a ‘Not Red’ classification indicative of a patient not at high-risk.

A knee anterior cruciate ligament (ACL) acute tear task can classify a radiology report relating to a patient's knee into a binary ‘Acute Tear’ classification or a ‘Not Acute Tear’ classification.

A knee ACL complete tear task can classify a radiology report relating to a patient's knee into a binary ‘Complete Tear’ classification or a ‘Non-complete Tear’ classification.

A neural foraminal stenosis task can classify a radiology report relating to a patient's cervical spine into a binary ‘Normal’ classification or an ‘Abnormal’ classification.

### Example Workflow

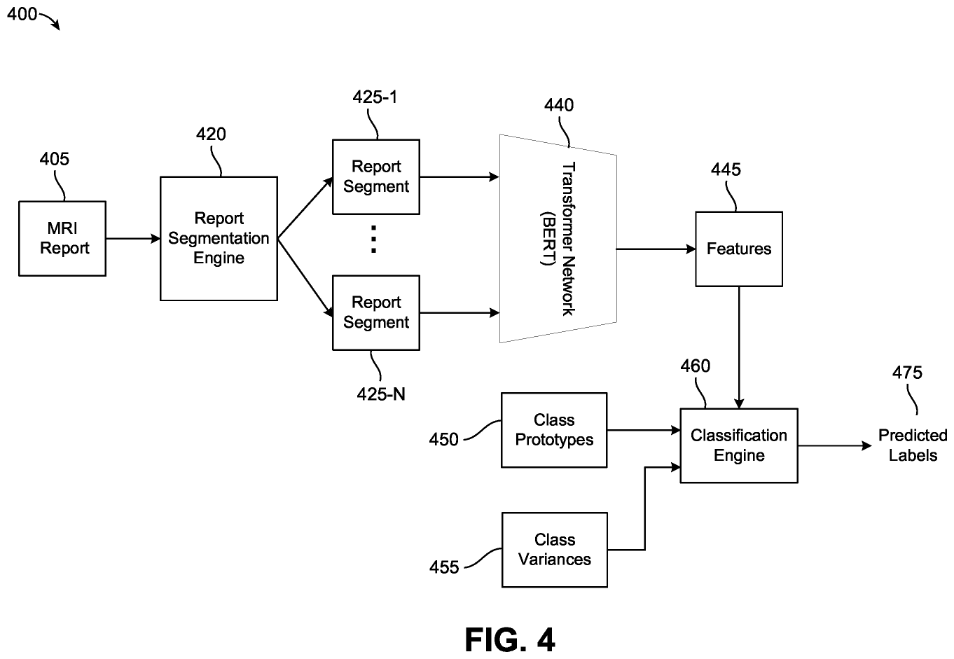

FIG. 4 is a diagram illustrating an example machine learning architecture and workflow 400 that can be used to perform medical NLP tasks (as described herein) using a variance-aware prototypical network.

As illustrated, one or more radiology reports 405 can be received as input. In some aspects, the input radiology report 405 can be an MRI report, as shown in FIG. 4, although various other types of radiology reports may also be utilized as input without departing from the scope of the present disclosure.

The input radiology report 405 (e.g., MRI report) can be first de-identified according to HIPAA regulations. For instance, the input radiology report 405 can be a de-identified report. The input radiology report 405 (de-identified or otherwise) can subsequently be passed through a sentence parser that splits the report into sentences. For instance, the sentence parses can be implemented using a report segmentation engine 420, which may include one or more machine learning networks for segmenting radiology report 405 into its constituent sentences.

The report segmentation engine 420 can generate as output a plurality of report segments 425-1, . . . , 425-N, where each of the report segments 425 includes at least a portion of the content of input radiology report 405. In some examples, each sentence corresponds to a separate segmentation instance. In other examples, a segmentation instance can include multiple sentences, or more than one sentence.

In one illustrative example, the machine learning workflow 400 can be implemented as a body part-specific workflow. In some embodiments, all reports. Irrespective of the particular body part to which the report (e.g., report 405) corresponds, are first de-identified according to HIPAA regulations. The de-identified report is then passed through a sentence parser (e.g., report segmentation engine 420) to parse the report into sentences (e.g., report segments 425-1, . . . , 425-N).

In some embodiments, a body-part specific custom data processor can be used to obtain the relevant text from a body-part specific radiology report, where the relevant text is used to predict the appropriate pathology severity.

For instance, if the input radiology report 405 is a lung report, the report segmentation engine 420 can be a lung-specific segmentation engine. In some embodiments, a lung-specific segmentation engine can be a rule-based regex configured to extract an ‘Impression’ section from the entire lung-specific input radiology report 405. The ‘Impression’ section is a summary of the report and contains all critical information such as number of lung nodules, size of lung nodules, potential of each lung nodule for malignancy, etc. The extracted text of the ‘Impression’ section of the lung radiology report 405 is used for a final (e.g., downstream) classification task performed by a classification engine 460 implementing the presently disclosed variance-aware prototypical network(s).

In another example, if the input radiology report 405 is a cervical spine report, the report segmentation engine 420 can be a cervical spine-specific segmentation engine. In some embodiments, the cervical spine-specific segmentation engine can be associated with a downstream task (e.g., implemented by classification engine 460 using one or more variance-aware prototypical networks) of predicting the severity of a neural foraminal stenosis for each MRI motion segment associated with the cervical spine radiology report 405. The motion segment is the smallest physiological motion unit of the spinal cord. In some aspects, breaking information down at the motion segment level can enable pathological findings to be correlated with clinical exam findings, a correlation which may inform future treatment interventions.

In one illustrative example, the transformer network 440 is a BERT-based named entity recognition (NER) machine learning model. In some aspects, the BERT transformer network 440 can receive as input the report segments 425-1, . . . , 425-N extracted from the cervical spine-=specific input radiology report 405, and may be used to identify the motion segment(s) referenced in each sentence of input radiology report 405. The BERT transformer network 440 may additionally identify all the sentences of the radiology report 405 containing a particular motion segment. For instance, all sentences referring to or containing the same (or particular) motion segment can be concatenated together.

In some cases, an additional rule-based logic can be used to assign, by BERT transformer network 440, motion segments to sentences that do not explicitly mention a motion segment (e.g., implicit references to a motion segment can be tagged with the explicit motion segment referred to implicitly in the sentence). The concatenated text can be included in the set of output features 445 generated by the BERT transformer network 440 and provided as an input to the variance-aware prototypical network-based classification engine 460. The features 445 can correspond to concatenated text at each motion segment.

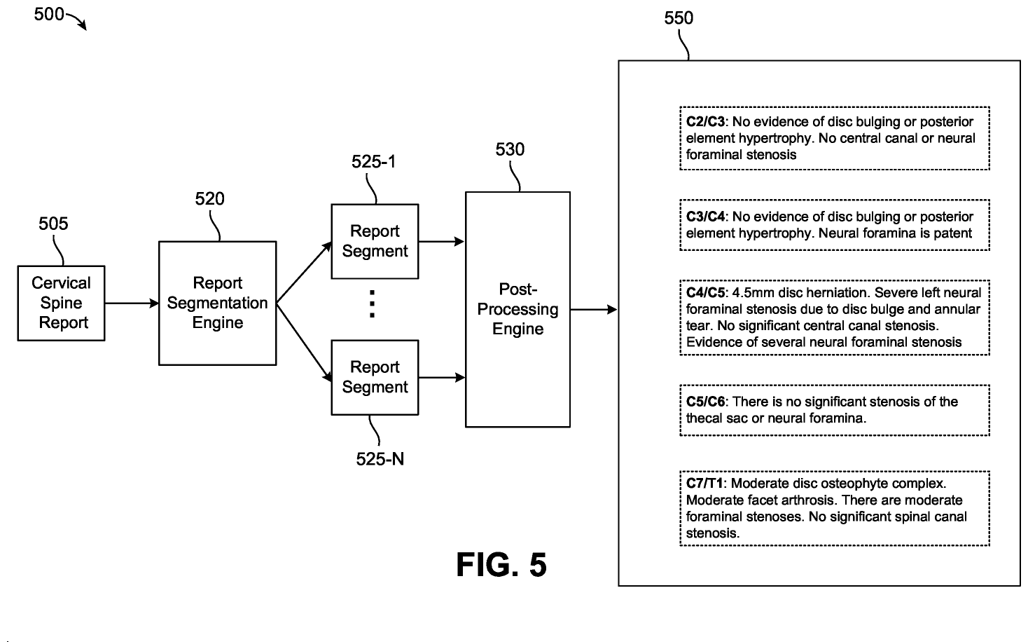

For instance, FIG. 5 is a diagram illustrating an example of report segmentation for cervical-spine specific input radiology reports 405. The cervical spine report 505 of FIG. 5 can be the same as the input radiology report 405 of FIG. 4. The report segmentation engine 520 can be a cervical-spine specific version or implementation of the report segmentation engine 420 of FIG. 4. The report segments 525-1, . . . , 525-N can be cervical spine-specific report segments (e.g., sentences) that are the same as or similar to the report segments 425-1, . . . , 425-N of FIG. 4 (e.g., can be cervical spine-specific report segments). The post-processing engine 530 of FIG. 5 can be the same as or similar to the post-processing engine 430 of FIG. 4. In some embodiments, post-processing engine 530 of FIG. 5 is a cervical spine-specific implementation of the post-processing engine 430 of FIG. 4.

As illustrated in FIG. 5, the output of post-processing engine 530 can comprise a plurality of concatenated text segments organized by the corresponding motion segment. The output 550 can include a first group of concatenated text segments extracted from input cervical spine report 505 where each text segment corresponds to the C2/C3 motion segment. The concatenated sentences can be consecutive or non-consecutive sentences in the underlying input cervical spine report 505. Similarly, output 550 includes a group of concatenated text segments for the C3/C4 motion segments, for the C4/C5 motion segments, for the C5/C6 motion segments, for the C7/T1 motion segments, etc.

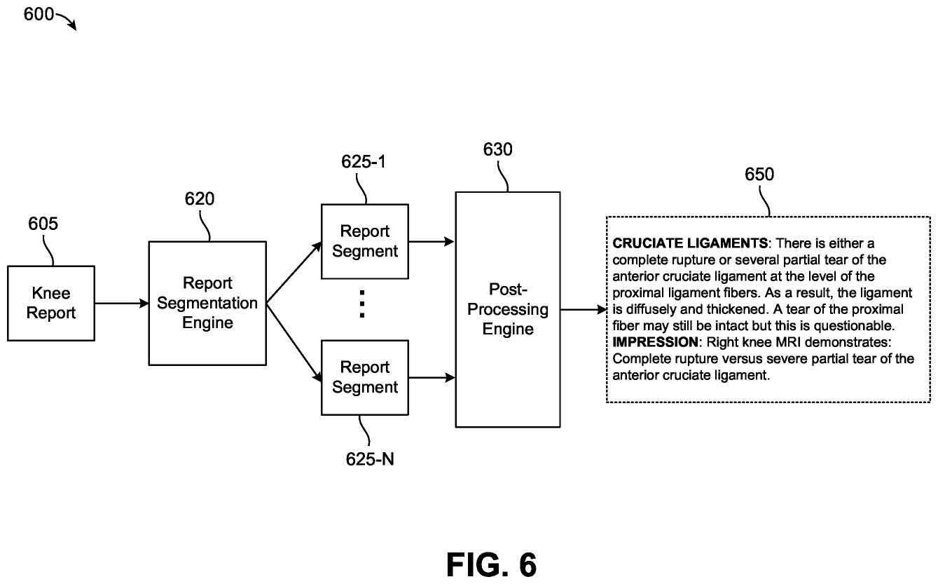

In yet another example, if the input radiology report 405 of FIG. 4 is a knee radiology report, the report segmentation engine 420 of FIG. 4 can be a knee-specific segmentation engine. For instance, FIG. 6 depicts an example architecture 600 corresponding to a knee-specific implementation of the architecture and workflow 400 of FIG. 4. The input knee radiology report 605 can be the same as or similar to the input radiology report 405 of FIG. 4 (e.g., a knee-specific radiology report). the report segmentation engine 620 can be a knee-specific implementation of the report segmentation engine 420 of FIG. 4. The report segments 625-1, . . . , 625-N can be knee-specific report segments extracted from the knee-specific input radiology report 605 of FIG. 6. The post-processing engine 630 can be a knee-specific post-processing engine implementation of the post-processing engine 430 of FIG. 4.

The output 650 of knee-specific workflow 600 (e.g., the output of post-processing engine 630) of FIG. 6 can be generated using a BERT-based NER model (e.g., the transformer network 440 of FIG. 4, the same as or similar to that described above with respect to FIGS. 4 and 5). In some embodiments, similar to the cervical dataset example of FIG. 5, the BERT-based NER model can be used to automatically tag sentences that mentioned the structure of importance in the knee anatomy (e.g., the anterior cruciate ligament (ACL)). In some aspects, all sentences that mention the structure of importance/interest (e.g., ACL) are grouped together in the output 650. In some cases, the output 650 groups together all sentences from knee-specific input radiology report 605 that mention the ACL. The grouped sentences of output 650 can be used to predict pathologies of importance, for instance using the variance-aware prototypical network-based machine learning classification engine 460 of FIG. 4. In one illustrative example, the output of classification engine 460 can be the predicted labels 475, where the predicted labels 475 are predicted pathology severities. The predicted labels 475 can be severities that are specific or otherwise correspond to the body-part specific pathology used to adjust the report segmentation engine 420 and post-processing engine to their body-part specific form as described above.

For instance, for the lung nodule task, the predicted labels 475 can be either the ‘Not Red’ or the ‘Red’ classification, as described above. For the knee ACL acute tear task, the predicted labels 475 can be either the ‘Not Acute Tear or ‘Acute Tear’ classification, as described above. For the knee ACL complete tear task, the predicted labels 475 can be either the ‘Not Complete Tear’ or ‘Complete Tear’ classification, as described above. For the neural foraminal stenosis task, the predicted labels 475 can be either the ‘Not Abnormal’ or ‘Abnormal’ classification, as described above.

In one illustrative example, the systems and techniques can implement a classification engine for predicting anatomical pathology severity using one or more variance-based prototypical networks. For instance, the classification engine 460 of FIG. 4 can include and/or be based on one or more variance-aware prototypical networks, as will be described in greater detail below.

Prototypical Networks or ProtoNets use an embedding function fo to encode each input (e.g., a query example) into an M-dimensional feature vector. For instance, the features 445 of FIG. 4 can correspond to the M-dimensional feature vector for a prototypical network. In some embodiments, the transformer network (e.g., BERT) 440 of FIG. 4 can be a feature generator or encoder associated with implementing a prototypical network.

A prototype is defined for every class c E L that is represented in a training dataset (e.g., the set L represents the known or seen classes from training of the prototypical network, such as by using an FSL-based approach over the L known classes). As noted previously, the class prototype c can be calculated as the mean vector of the embedded support data samples for the given class:

$${υ}_{c} = \frac{1}{\vert {S}_{c} \vert} {\sum }_{( {x}_{i} , {y}_{i} ) ∈ {S}_{c}} {f}_{θ} ( {x}_{i} ) \qquad Eq . ( 1 )$$

Here, v_{c} is the prototype representation for the given class c. S_{c} represents the number of samples for the support set S (e.g., the number of embedded support data samples for each given class c). f_{θ} is an encoder (e.g., machine learning encoder or feature generator, such as the transformer network (BERT) 440 of FIG. 4) with parameters θ. As seen in Eq. (1), the prototype representation, v_{c}, for each class c can be determined as the mean (e.g., average) of the embeddings of the support set S_{c} provided for the class c. Note that in the example of Eq. (1), the variable xᵢ is associated with a query and the variable yᵢ is associated with the label or classification assigned to (e.g., determined for) the query xᵢ.

The distribution over classes for a given test input x (e.g., the query xᵢ) can be determined as a softmax over the inverse of distances between the test data embedding and prototype vectors. In other words, given a query x and based on the prototypes v_{c}, the prototypical network can determine or otherwise obtain a probability distribution over the c known classes that were seen during training of the prototypical network:

$$\begin{matrix}P ( y = c \vert x ) = softmax ( - d ( {f}_{θ} ( x ) , {υ}_{c} ) ) \\ = \frac{exp ( - d ( {f}_{θ} ( x ) , {υ}_{c} ) )}{{\sum }_{{c}^{′} ∈ L} exp ( - d ( {f}_{θ} ( x ) , {υ}_{{c}^{′}} ) )}\end{matrix} \qquad Eq . ( 2 )$$

Here, d(⋅) is a distance metric for characterizing the distance between the query embedding f_{θ}(x) and a prototype v_{c}. In some aspects, d(⋅) can be any (differentiable) distance function. Specific examples of the distance metrics utilized by the systems and techniques described herein are explained in greater detail below. In some cases, a conventional approach to implementing a prototypical network is to use a Euclidean distance (e.g., d(z, z′)=∥z−z′∥²), as has been noted previously above.

In some aspects, the prototypical network associated with or otherwise implementing Eqs. (1) and (2) can be trained based on minimizing a negative log-probability, which can be given as the negative log-probability of the true class c. In one illustrative example, the prototypical network can be trained based on a loss function given as the negative log-likelihood:

[symbol]

(θ)=−log P_{θ}(y=c|x) Eq. (3)

In one illustrative example, the probability distribution of Eq. (2) (e.g., the conditional probability distribution of the query x over all of the classes c) can be used to classify the input/query x by determining the distance (e.g., using the distance metric d(⋅)) between the query example x and the prototypical representation v_{c} for each class. For example, if the input query example x is closest to class number three (e.g., of the N classes), then a relatively high probability can be determined for class three and a relatively lower probability for the remaining N−1 classes. For example, these probabilities can be determined based on Eq. (2), which itself can be determined based on the distance metric d(⋅).

ProtoNets are simple and easy to train and deploy. The mean of the embeddings calculated for a support set S_{c} provided for a given class c is used to capture the entire conditional distribution P(y=c|x), thus losing a lot of information about the underlying distribution. ProtoNets may be improved by taking into account the above observation relating to the underlying conditional distribution and associated information loss.

Aspects of the present disclosure extend ProtoNets by incorporating the variance (i.e., 2nd moment) of the conditional probability distribution P(y=c|x). In one illustrative example, the systems and techniques described herein can use distributional distance (e.g., a 2-Wasserstein metric) as the distance metric d(⋅) of Eq. (2), thereby directly generalizing existing or vanilla ProtoNets (e.g., conventional prototypical networks).

### Variance-Aware ProtoNets

In some embodiments, a variance-aware prototypical network can be implemented based on modeling each conditional distribution (e.g., the distribution(s) P(y=c|x)) as a Gaussian. In such examples, the variance-aware prototypical network can be modified in order to match a query example (e.g., a query x) with a Gaussian distribution that replaces or models the conventional conditional distribution P(y=c|x).

One approach is to treat the query example x as a Dirac distribution. Recall that the Wasserstein-Bures metric between Gaussians (mᵢ, Σᵢ) is given by:

d²=∥mᵢ−m₂∥²+Tr(Σ₁+Σ₂−2(Σ₁^{1/2}Σ₂Σ₁^{1/2})^{1/2} Eq. (3)

Given xᵢ, yᵢ∈S_{c} (e.g., given a query example and corresponding classification label that are elements of the support set S_{c}), the corresponding mean m_{c} and covariance matrix Σ_{c} are computed. Using the mean m_{c} and covariance matrix Σ_{c}, the computation of Wasserstein distance between a Gaussian (e.g., modeling the conditional probability distribution for the prototypical network) and a query vector q (e.g., a Dirac) can be determined using the below simplification of Eq. (3):

d²=∥m_{c}−q∥²+Tr(Σ_{c}) Eq. (4)

The formulation of Eq. (4) above demonstrates that the prototypical network conditional distribution P(y=c|x)) can be simplified as a Gaussian with a diagonal covariance matrix Σ_{c}. The simplification to a Gaussian with a diagonal covariance matrix reduces the space complexity to store the covariance matrix from O(n²) to O(n).

It is additionally noted that the approach above of Eqs. (1)-(4) can be seen as a direct generalization of vanilla prototypical networks (e.g., existing or conventional prototypical networks), as the vanilla prototypical networks can be interpreted as computing the Wasserstein distance (e.g., simple L₂ distance) between two Dirac distributions (e.g., the prototype/mean of the conditional distribution and the query sample).

In another illustrative example, the systems and techniques described herein additionally contemplate another variant of the approach(es) described above, for example based on using an Isotropic Gaussian variant that averages over the diagonal entries of Σ_{c}, i.e., using

$$α = \frac{1}{n} {( {\sum }_{c} )}_{i i}$$

and redefining the covariance matrix Σ_{c} as Σ_{c}=αIₙ.

In some embodiments, the variance-aware prototypical networks described herein can be trained based on regularizing the negative log likelihood loss of Eq. (3), above, to prevent the variance term from increasing drastically or uncontrollably (e.g., from blowing up). The variance term, in the below re-formulation of Eq. (2), replaces the conditional distribution term P(y=c|x) of Eq. (2). The modified loss function can be provided as:

[symbol]

(θ)= [symbol] (θ)+λ/·ways∥Σ_{c}∥_{F} Eq. (5)

Here, ∥⋅∥_{F} is the Frobenius norm, and may be applied to the variance matrix (e.g., the covariance matrix Σ_{c}). The (Frobenius) norm of the variance matrix is averaged over all the classes in a given meta-batch, represented in Eq. (5) as the term “#ways”—recalling that an N-way few-shot learning is performed over N different classes. In other words, the term “#ways” can be the same as the number of classes c and/or the number of prototypes v_{c} used variously in the formulations above of Eqs. (1)-(4).

The extra or additional regularization term is designed to encourage the input/query examples provided to the variance-aware prototypical network to be close to the appropriate cluster centroid (e.g., a prototype representation). This regularization term can also be seen as an entropic regularization term, i.e. up to a factor as the exponential of KL(p∥q), where p=N(m_{c}, Σ_{c}) and q N(m_{c},I). This is a type of entropy-regularized Wasserstein distance.

### Experimental Results

In one example, the example experiments summarized below may be run on one V100 16 GB GPU using PyTorch and HuggingFace libraries, although it is noted these example experiments are provided for purposes of illustration only and are not intended to be construed as limiting. BERT-base, Clinical BERT, and PubMed-BERT are used as backbone models. Adapters can be applied to each of these backbone models. While training adapter-based models, the BERT weights are frozen and only the adapter weights are updated, thus requiring less resources to train. In other words, the features from these deep pre-trained models may be reused. The presently disclosed methods are compared and analyzed against various benchmarks and baselines. The results for the BERT-base and the Clinical BERT backbones are summarized below in Table 1 and Table 2:

TABLE 1
Example results on a meta-validation dataset. In this example,
1,000 tasks were sampled with 4-way 8-shot (e.g., N-shot M-
way formulation with N = 4 and M = 8) and 16-support
classification. Each experiment was replicated over 10 random seeds.

| Backbone | Methods | Accuracy |
|---|---|---|
| BERT-base | Vanilla ProtoNet | 86.3 ± 1.2 |
| | Big ProtoNet | 87.8 ± .9 |
| | Leopard | 81.4 ± 9.7 |
| | ProtoNet w/Isotropic Gaussian | 88.7 ± 1.4 |
| | ProtoNet w/Isotropic Gaussian + reg | 89.5 ± .8 |
| | Variance Aware ProtoNet (ours) | 88.9 ± 1.5 |
| | Variance Aware ProtoNet + reg (ours) | 90.1 ± .9 |
| BERT-base | Vanilla ProtoNet | 85.6 ± 1.3 |
| w/Adapters | Big ProtoNet | 87.1 ± 1.1 |
| | ProtoNet w/Isotropic Gaussian | 87.8 ± .8 |
| | ProtoNet w/Isotropic Gaussian + reg | 88.6 ± .7 |
| | Variance Aware ProtoNet (ours) | 88.1 ± 1.2 |
| | Variance Aware ProtoNet + reg (ours) | 89.7 ± .8 |
| Clinical BERT | Vanilla ProtoNet | 87.4 ± 1.3 |
| | Big ProtoNet | 88.5 ± 1.1 |
| | Leopard | 82.2 ± 9.8 |
| | ProtoNet w/Isotropic Gaussian | 89.6 ± 1.2 |
| | ProtoNet w/Isotropic Canssian + reg | 90.1 ± .8 |
| | Variance Aware ProtoNet (ours) | 89.9 ± 1.1 |
| | Variance Aware ProtoNet + reg (ours) | 90.9 ± .8 |
| Clinical BERT | Vanilla ProtoNet | 86.8 ± .9 |
| w/Adapters | Big ProtoNet | 87.9 ± 1.1 |
| | ProtoNet w/Isotropic Gaussian | 88.4 ± 1.3 |
| | ProtoNet w/Isotropic Gaussian + reg | 89.1 ± .9 |
| | Variance Aware ProtoNet (ours) | 88.7 ± 1.1 |
| | Variance Aware ProtoNet + reg (ours) | 89.5 ± .9 |

TABLE 2
Examples of successful application of few-shot models with BERT-base and
Clinical BERT backbones in straight-up, supervised classification tasks.

| | | | Knee | Knee | |
|---|---|---|---|---|---|
| Backbone | Methods | Foraminal | (Acute Tear vs Not) | (Complete tear vs Not) | Lung |
| BERT-base | Baseline | .24 | .29 | .32 | .19 |
| | Multi-Task | .29 | .34 | .41 | .27 |
| | Vanilla ProtoNet | .75 | .71 | .66 | .65 |
| | Big ProtoNet | .57 | .58 | .53 | .6 |
| | Leopard | .63 | .72 | .61 | .41 |
| | ProtoNet w/Isotropic Gaussian | .77 | .72 | .69 | .68 |
| | ProtoNet w/Isotropic Gaussian + reg | .78 | .76 | .71 | .7 |
| | Variance Aware ProtoNet (ours) | .79 | .78 | .73 | .72 |
| | Variance Aware ProtoNet + reg (ours) | .81 | .8 | .76 | .75 |
| BERT-base | Baseline | .28 | .32 | .4 | .25 |
| w/Adapters | Multi-Task | .32 | .35 | .44 | .29 |
| | Vanilla ProtoNet | .74 | .73 | .65 | .67 |
| | Big ProtoNet | .58 | .59 | .55 | .61 |
| | ProtoNet w/Isotropic Gaussian | .78 | .71 | .67 | .69 |
| | ProtoNet w/Isotropic Gaussian + reg | .8 | .74 | .72 | .74 |
| | Variance Aware ProtoNet (ours) | .8 | .74 | .72 | .74 |
| | Variance Aware ProtoNet + reg (ours) | .82 | .77 | .77 | .78 |
| Clinical BERT | Baseline | .31 | .37 | .42 | .28 |
| | Multi-Task | .34 | .45 | .47 | .38 |
| | Vanilla ProtoNet | .77 | .72 | .68 | .66 |
| | Big ProtoNet | .57 | .59 | .53 | .61 |
| | Leopard | .74 | .78 | .77 | .62 |
| | ProtoNet w/Isotropic Gaussian | .78 | .74 | .71 | .68 |
| | ProtoNot w/Isotropic Gaussian + reg | .8 | .76 | .74 | .71 |
| | Variance Aware ProtoNet (ours) | .82 | .79 | .76 | .74 |
| | Variance Aware ProtoNet + reg (ours) | .84 | .81 | .79 | .76 |
| Clinical BERT | Baseline | .35 | .42 | .45 | .33 |
| w/Adapters | Multi-Task | .37 | .45 | .49 | .37 |
| | Vanilla ProtoNet | .76 | .74 | .7 | .67 |
| | Big ProtoNet | .58 | .6 | .57 | .62 |
| | ProtoNet w/Isotropic Gaussian | .79 | .76 | .72 | .7 |
| | ProtoNet w/Isotropic Gaussian + reg | .81 | .77 | .73 | .72 |
| | Variance Aware ProtoNet (ours) | .83 | .81 | .76 | .73 |
| | Variance Aware ProtoNet + reg (ours) | .85 | .82 | .81 | .77 |

To prevent overfitting on the test set, the example experiments proceeded based on selecting the best model from each of the experiments summarized in Tables 1 and 2, for instance with the “best” model selection performed based on the meta-validation accuracy information (also summarized in Tables 1 and 2). The selected best model was subsequently applied to the example downstream classification tasks described herein for severity prediction/classification of body-part specific anatomical pathology detections. It is noted that these downstream tasks are significantly different than the tasks represented in the training data used to perform training in the few-shot regime these models are trained in (e.g., significantly different than the tasks represented in the FSL training dataset).

For each of the downstream tasks, the example experiments can be performed based on training BERT models on each task (e.g., a lung-specific BERT model, a knee ACL-specific BERT model (or an acute tear knee ACL-specific BERT model and a complete tear knee ACL-specific BERT model), and a cervical-spine specific BERT model), as well as based on training a multi-tasking model, where the BERT models and the multi-tasking model are trained to provide additional baselines.

In the example experiments, PubMedBERT consistently outperforms BERT-base and Clinical BERT by an average of 5 points and 3 points respectively. The improved performance may be attributable to the domain-specific vocabulary of PubMedBERT. Although Clinical BERT is pre-trained on MIMIC-III, Clinical BERT still shares the same vocabulary as BERT-base.

ProtoNet-BERT shows better performance and faster convergence rates during training and validation (see e.g., Table 4), but it is outperformed by ProtoNet-AdapterBERT which has fewer orders of magnitude of parameters to learn (see e.g., Table 3):

TABLE 3
Examples of successful applications of Few-Shot machine learning
models in straight-up, supervised classification tasks.

| | | | Knee | Knee | |
|---|---|---|---|---|---|
| Backbone | Methods | Foraminal | (Acute Tear vs Not) | (Complete tear vs Not) | Lung |
| PubMedBERT | Baseline | .38 | .44 | .49 | .36 |
| | Multi-Task | .41 | .47 | .52 | .39 |
| | Vanilla ProtoNet | .79 | .73 | .6 | .68 |
| | Big ProtoNet | .58 | .59 | .51 | .64 |
| | Leopard | .84 | .78 | .80 | .74 |
| | ProtoNet w/Isotropic Gaussian | .81 | .74 | .76 | .69 |
| | ProtoNet w/Isotropic Gaussian + reg | .83 | .76 | .77 | .73 |
| | Variance Aware ProtoNet (ours) | .84 | .78 | .79 | .76 |
| | Variance Aware ProtoNet + reg (ours) | .86 | .81 | .84 | .8 |
| PubMedBERT | Baseline | .42 | .47 | .51 | .41 |
| w/Adapters | Multi-Task | .44 | .49 | .53 | .43 |
| | Vanilla ProtoNet | .78 | .71 | .69 | .66 |
| | Big ProtoNet | .59 | .57 | .54 | .67 |
| | ProtoNet w/Isotropic Gaussian | .83 | .75 | .78 | .72 |
| | ProtoNet w/Isotropic Gaussian + reg | .89 | .8 | .86 | .77 |
| | Variance Aware ProtoNet (ours) | .87 | .77 | .81 | .74 |
| | Variance Aware ProtoNet + reg (ours) | .91 | .82 | .89 | .78 |

TABLE 4
Example experimental results on the meta-validation dataset. 1,000
tasks were sampled with 4-way 8-shot and 16-support classification.
Each experiment was replicated over 10 random seeds.

| Backbone | Methods | Accuracy |
|---|---|---|
| PubMedBERT | Vanilla ProtoNet | 89.1 ± 1.1 |
| | Big ProtoNet | 90.8 ± 1.2 |
| | Leopard | 85.1 ± 9.2 |
| | ProtoNet w/Isotropic Gaussian | 90.2 ± 1.4 |
| | ProtoNet w/Isotropic Gaussian + reg | 92.1 ± .8 |
| | Variance Aware ProtoNet (ours) | 91.5 ± 1.3 |
| | Variance Aware ProtoNet + reg (ours) | 92.9 ± .9 |
| PubMedBERT | Vanilla ProtoNet | 88.3 ± 1.4 |
| w/Adapters | Big ProtoNets | 89.4 ± 1.2 |
| | ProtoNet w/Isotropic Gaussian | 89.8 ± 1.4 |
| | ProtoNet w/Isotropic Gaussian + reg | 90.9 ± .7 |
| | Variance Aware ProtoNet (ours) | 90.5 ± 1.3 |
| | Variance Aware ProtoNet + reg (ours) | 91.2 ± .8 |

In some cases, ProtoNet-BERT may be more vulnerable to overfitting on the meta-training tasks than the ProtoNet-AdapterBERT. Finally, it is noted that even though Big ProtoNets work well on meta-validation, they fail on some of the presently discussed downstream classification tasks. This may potentially be due to the fact that Big ProtoNets are encouraged to have large radii; this has the potential to become a bottleneck in downstream tasks where the data distribution is highly imbalanced causing the spherical Gaussians to overlap. In some aspects, doing the exact opposite (e.g., constricting the norms of the covariance matrix), tends to produce best results on our downstream tasks. Finally, it is noted that instead of using the entire validation set to compute the class distribution, the systems and techniques described herein may be implemented based on choosing k shots from the validation set to compute the class distribution

The presently disclosed variance-aware prototypical networks with variance regularization using BERT-base+Adapter is also validated on 13 public benchmark datasets. For the models and datasets marked with an asterisk (*) in Table 5, the results reported in (Bansal et al., 2020a) are used, and for those datasets, the techniques from (Wang et al., 2021) are used to generate the example experimental results for ProtoNet with Bottleneck Adapters. The presently disclosed systems and techniques outperform Leopard by 5, 3 and 2 points on 4, 8 and 16 shots, respectively.

TABLE 5
Example experimental results on benchmark text datasets on a wide range of tasks, from NLI,
sentiment analysis, and text classification. In the example experimental results, the presently
disclosed variance-aware prototypical network uses BERT-base with bottleneck Adapters.

| | | | MT- | | | ProtoNet + | Variance Aware |
|---|---|---|---|---|---|---|---|
| Shots | Dataset | BERT* | BERT* | Leopard | ProtoNet | Adapter | ProtoNet (Ours) |
| 4 | airline | 42.76 ± | 46.29 ± | 54.95 ± | 65.39 ± | 65.33 ± | 62.67 ± |
| | | 13.50 | 12.26 | 11.81 | 12.73 | 7.95 | 11.18 |
| | disaster | 55.73 ± | 50.61 ± | 51.45 ± | 54.01 ± | 53.48 ± | 53.89 ± |
| | | 10.29 | 8.33 | 4.25 | 2.9 | 4.76 | 3.79 |
| | emotion | 9.20 ± | 9.84 ± | 11.71 ± | 11.69 ± | 12.52 ± | 15.15 ± |
| | | 3.22 | 2.14 | 2.16 | 1.87 | 1.32 | 4.19 |
| | political_audience | 51.89 ± | 51.53 ± | 52.60 ± | 52.77 ± | 51.88 ± | 52.5 ± |
| | | 1.72 | 1.80 | 3.51 | 5.86 | 6.37 | 6.45 |
| | sentiment_kitchen* | 56.93 ± | 60.53 ± | 78.35 ± | 62.71 ± | 83.13 ± | 84.16 ± |
| | | 7.10 | 9.25 | 18.36 | 9.53 | 0.96 | 1.37 |
| | political_bias | 54.57 ± | 54.66 ± | 60.49 ± | 58.26 ± | 61.72 ± | 59.39 ± |
| | | 5.02 | 3.74 | 6.66 | 10.42 | 5.65 | 6.18 |
| | rating_electronics* | 39.27 ± | 41.20 ± | 51.71 ± | 37.40 ± | 53.81 ± | 55.49 ± |
| | | 10.15 | 0.69 | 7.20 | 3.72 | 6.01 | 5.42 |
| | political_message | 15.64 ± | 14.49 ± | 15.69 ± | 17.82 ± | 20.98 ± | 19.28 ± |
| | | 2.73 | 1.75 | 1.57 | 1.33 | 1.69 | .91 |
| | sentiment_books* | 54.81 ± | 64.93 ± | 82.54 ± | 73.15 ± | 83.88 ± | 84.95 ± |
| | | 3.75 | 8.65 | 1.33 | 5.85 | 0.55 | 1.72 |
| | rating_books* | 39.42 ± | 38.97 ± | 48.44 ± | 54.92 ± | 59.20 ± | 66.18 ± |
| | | 07.22 | 13.27 | 7.43 | 6.18 | 7.26 | 7.89 |
| | rating_dvd* | 32.22 ± | 41.23 ± | 49.76 ± | 47.73 ± | 50.20 ± | 52.59 ± |
| | | 08.72 | 10.98 | 9.80 | 6.20 | 10.26 | 14.09 |
| | rating_kitchen | 34.76 ± | 36.77 ± | 50.21 ± | 58.47 ± | 55.99 ± | 59.39 ± |
| | | 11.2 | 10.62 | 9.63 | 11.12 | 9.85 | 8.79 |
| | scitail* | 58.53 ± | 63.97 ± | 69.50 ± | 76.27 ± | 77.84 ± | 79.16 ± |
| | | 09.74 | 14.36 | 9.56 | 4.26 | 2.61 | 2.54 |
| | Average | 41.98 | 44.23 | 52.11 | 51.58 | 56.15 | 57.29 |
| 8 | airline | 38.00 ± | 49.81 ± | 61.44 ± | 69.14 ± | 69.37 ± | 69.31 ± |
| | | 17.06 | 10.86 | 3.90 | 4.84 | 2.46 | 2.43 |
| | disaster | 56.31 ± | 54.93 ± | 55.96 ± | 54.48 ± | 53.85 ± | 55.19 ± |
| | | 9.57 | 7.88 | 3.58 | 3.17 | 3.03 | 2.77 |
| | emotion | 8.21 ± | 11.21 ± | 12.90 ± | 13.10 ± | 13.87 ± | 15.1 ± |
| | | 2.12 | 2.11 | 1.63 | 2.64 | 1.82 | 3.58 |
| | political_audience | 52.80 ± | 54.34 ± | 54.31 ± | 55.17 ± | 53.08 ± | 53.82 ± |
| | | 2.72 | 2.88 | 3.95 | 4.28 | 6.08 | 4.13 |
| | sentiment_kitchen* | 57.13 ± | 69.66 ± | 84.88 ± | 70.19 ± | 83.48 ± | 84.69 ± |
| | | 6.60 | 8.05 | 1.12 | 6.42 | 0.44 | .8 |
| | political_bias | 56.15 ± | 54.79 ± | 61.74 ± | 63.22 ± | 65.36 ± | 64.09 ± |
| | | 3.75 | 4.19 | 6.73 | 1.96 | 2.03 | .58 |
| 8 | rating_electronics* | 28.74 ± | 45.41 ± | 54.78 ± | 43.64 ± | 56.97 ± | 60.24 ± |
| | | 08.22 | 09.49 | 6.48 | 7.31 | 3.19 | 2.62 |
| | political_message | 13.38 ± | 15.24 ± | 18.02 ± | 20.40 ± | 21.64 ± | 20.44 ± |
| | | 1.74 | 2.81 | 2.32 | 1.12 | 1.72 | 1.17 |
| | sentiment_books* | 53.54 ± | 67.38 ± | 83.03 ± | 75.46 ± | 83.9 ± | 84.68 ± |
| | | 5.17 | 9.78 | 1.28 | 6.87 | 0.39 | .85 |
| | rating_books* | 39.55 ± | 46.77 ± | 59.16 ± | 52.13 ± | 61.74 ± | 65.54 |
| | | 10.01 | 14.12 | 4.13 | 4.79 | 6.83 | 6.78 |
| | rating_dvd* | 36.35 ± | 45.24 ± | 53.28 ± | 47.11 ± | 53.25 ± | 53.83 ± |
| | | 12.50 | 9.76 | 4.66 | 4.00 | 7.47 | 10.46 |
| | rating_kitchen | 34.49 ± | 47.98 ± | 53.72 ± | 57.08 ± | 56.27 ± | 56.68 ± |
| | | 8.72 | 9.73 | 10.31 | 11.54 | 10.70 | 11.21 |
| | scitail* | 57.93 ± | 68.24 ± | 75.00 ± | 78.27 ± | 80.41 ± | 80.57 ± |
| | | 10.70 | 10.33 | 2.42 | 0.98 | 1.05 | .48 |
| | Average | 40.97 | 48.54 | 56.02 | 53.8 | 57.94 | 58.78 |
| 16 | airline | 58.01 ± | 57.25 ± | 62.15 ± | 71.06 ± | 9.83 ± | 69.9 ± |
| | | 8.23 | 9.90 | 5.56 | 1.60 | 1.80 | 1.06 |
| | disaster | 64.52 ± | 60.70 ± | 61.32 ± | 55.30 ± | 57.38 ± | 60.14 ± |
| | | 8.93 | 6.05 | 2.83 | 2.68 | 5.25 | 5.36 |
| | emotion | 13.43 ± | 12.75 ± | 13.38 ± | 12.81 ± | 14.11 ± | 13.55 ± |
| | | 2.51 | 2.04 | 2.20 | 41.21 | 1.12 | 3.51 |
| | political_audience | 58.45 ± | 55.14 ± | 57.71 ± | 56.16 ± | 57.23 ± | 56.36 ± |
| | | 4.98 | 4.57 | 3.52 | 2.81 | 2.77 | 2.29 |
| | sentiment_kitchen* | 68.88 ± | 77.37 ± | 85.27 ± | 71.83 ± | 83.72 ± | 84.93 ± |
| | | 3.39 | 6.74 | 01.31 | 5.94 | 0.30 | .49 |
| | political_bias | 60.96 ± | 60.30 ± | 65.08 ± | 61.98 ± | 65.38 ± | 63.97 ± |
| | | 4.25 | 3.26 | 2.14 | 6.89 | 1.71 | 2.49 |
| | rating_electronics* | 45.48 ± | 47.29 ± | 58.69 ± | 44.83 ± | 56.62 ± | 61.01 ± |
| | | 06.13 | 10.55 | 2.41 | 5.96 | 5.62 | 1.54 |
| | political_message | 20.67 ± | 19.20 ± | 18.07 ± | 21.36 ± | 24.00 ± | 22.49 ± |
| | | 3.89 | 2.20 | 2.41 | 0.86 | 1.39 | 1.31 |
| | sentiment_books* | 65.56 ± | 69.65 ± | 83.33 ± | 77.26 ± | 83.92 ± | 84.91 ± |
| | | 4.12 | 8.94 | 0.79 | 3.27 | 0.41 | 0.66 |
| | rating_books* | 43.08 ± | 51.68 ± | 61.02 ± | 57.28 ± | 64.75 ± | 67.34 ± |
| | | 11.78 | 11.27 | 4.19 | 4.57 | 4.27 | 7.52 |
| | rating_dvd* | 42.79 ± | 45.19 ± | 53.52 ± | 48.39 ± | 55.08 ± | 56.63 ± |
| | | 10.18 | 11.56 | 4.77 | 3.74 | 4.92 | 6.11 |
| | rating_kitchen | 47.94 ± | 53.79 ± | 57.00 ± | 61.00 ± | 59.45 ± | 58.34 ± |
| | | 8.28 | 9.47 | 8.69 | 9.17 | 8.33 | 11.72 |
| | scitail* | 65.66 ± | 75.35 ± | 77.03 ± | 78.59 ± | 80.27 ± | 80.89 ± |
| | | 06.82 | 04.80 | 1.82 | 0.48 | .75 | .23 |
| | Average | 50.42 | 52.74 | 57.97 | 55.22 | 59.36 | 60.04 |

### Deployment

Based on the results depicted above in Table 3, the systems and techniques can be implemented based on deploying the regularized variance-aware ProtoNet with Adapter-PubMedBERT. In one illustrative example, the variance-aware ProtoNet pipeline is deployed on AWS using a single p3.2x instance housed with one NVIDIA V100 GPU. The main pipeline components include 1) a body-part specific report segmentation engine (e.g., such as the body-part specific report segmentation engine 420 of FIG. 4, the cervical spine-specific report segmentation engine 520 of FIG. 5, and/or the knee-specific report segmentation engine 620 of FIG. 6); 2) the PubMedBERT backbone with adapters (e.g., the transformer network (BERT) 440 of FIG. 4); and 3) a dictionary of the class prototypes (e.g., v_{c}) and the class variances (e.g., Σ_{c}) for all classes (e.g., c) in the datasets.

On inference, requests sent to the pipeline include a body part which the pipeline utilizes to load up the relevant body part-specific report segmentation engine, class prototypes (e.g., class prototypes 450 of FIG. 4), and variances (e.g., class variances 455 of FIG. 4). A report (e.g., input radiology report) is then ingested by the pipeline, parsed by a sentencizer (e.g., the body part-specific segmentation engine), grouped into segments according to its body part specific segmentation (e.g., grouped into the segments 425-1, . . . , 425-N of FIG. 4; the segments 525-1, . . . 525-N of FIG. 5; the segments 625-1, . . . , 625-N of FIG. 6; etc.), and then passed to the model.

A class probability and labels (e.g., predicted labels 475 of FIG. 4) are inferred after computing the Wasserstein distance between the text embedding (e.g., features 445 of FIG. 4) and the appropriate class distributions (e.g., class prototypes 450 and/or class variances 455 of FIG. 4). The predicted probabilities (e.g., predicted labels 475 of FIG. 4) and pipeline metadata are written out to an AWS Redshift database cluster. The entire pipeline is orchestrated in batch mode with a large enough batch size to maximize the GPU capacity resulting in an average latency of 68 ms/report.

### Monitoring

In some aspects, BERT embeddings are highly anisotropic. In some examples, the same is true for the presently disclosed meta-learned models as well. This observation can be advantageously utilized to monitor out-of-distribution (OOD) cases.

In some embodiments, for each class in a given dataset, the systems and techniques can pick the top k-dimensions (e.g., where k is a hyperparameter) of maximum variance. The union of these indices can be determined. The indices may be referred to as the set of dataset indices (e.g., the indices that explain the variance among all classes in the dataset). For any given query example, the absolute difference (\vec{d}ⱼ) can be computed between the given query example and its embedding vector (\vec{q}ⱼ) and class centroids (\vec{v}ⱼ), i.e. the i-th coordinate \vec{d}ⱼ:\vec{d}ⱼ_{ᵢ}=|\vec{q}ⱼ−\vec{v}ⱼ_{ᵢ}|

The top k dimensions of the each of these dⱼ are then selected. In one illustrative example, an OOD metric referred to as Average Variance Indices (AVI_k) is described herein, and may be determined or otherwise calculated by the overlap between the top-k difference vector indices and the top-k dataset indices, i.e.,

$$AVI\_k := \frac{\vert {⋃}_{j = 1}^{c} top - k ( \tilde{{d}_{j}} ) \vert}{dataset indices} ,$$

where c is the number of classes. For example, in the case of the lung dataset: The text “The heart is normal in size. There is no pericardial effusion. The ascending aorta is nonaneurysmal. No intimal flap identified to suggest aortic dissection. The main pulmonary artery is enlarged” shows an AVI_10 score of 0.79, whereas the text “L1L2: There is no disc herniation in lumbar spine” gives a score of 0.31. As part of the pipeline monitoring implementation, reports can be thresholded with an AVI_10<0.5 to further investigate if the report is OOD.

### CONCLUSION

Described herein are systems and techniques for implementing an extension of Prototypical Networks in which Wasserstein distances are used as the distance metric between the embeddings calculated for a query input and the various class prototypes v_{c} determined for and used during training of the prototypical network (i.e., instead of the distance metric being based on cosine and/or Euclidean distance, as in conventional prototypical networks).

The systems and techniques are further seen to introduce a regularization term to encourage the class examples to be clustered closely to the class prototype. By training the presently disclosed variance-aware ProtoNets models on a label rich dataset (e.g., in this example, shoulder MRI reports), successful downstream performance is shown on a variety of labels on MRI reports on different body parts. Since the same model weights are reused for all tasks, a single model is deployed, thereby allowing significant savings in inference costs and computational complexity.

Moreover, the systems and techniques use adapters in the variance-aware ProtoNets models, thereby allowing tuning to be performed for only a small number of parameters (e.g., about 10 million parameters) resulting in huge training cost savings. Extensive experiments were conducted and are described above relating to validation of the presently disclosed systems and techniques for variance-aware ProtoNets, with validation performed on 13 public datasets and shown to outperform strong baselines like ProtoNets and Leopard. In some cases, the dataset statistics (e.g., which are already pre-computed) can be leveraged to define an OOD detection metric called Average Variance Indices (AVI) to identify potential OOD cases.

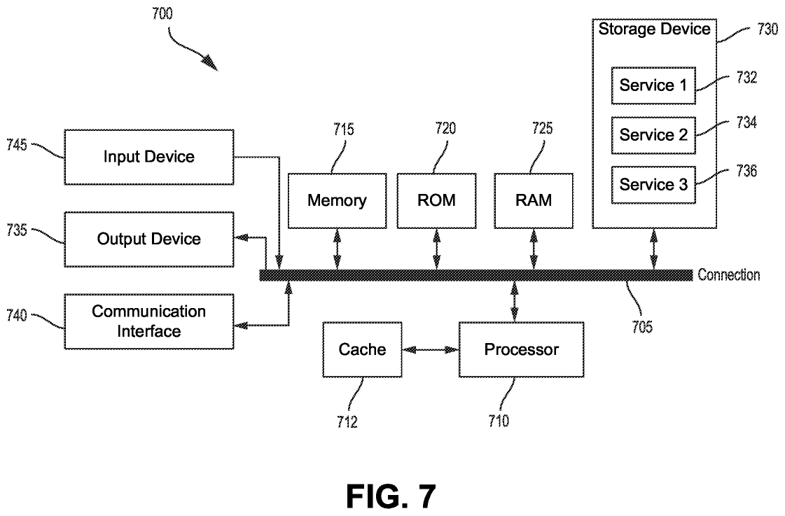

FIG. 7 illustrates an example computing device architecture 700 of an example computing device which can implement the various techniques described herein. In some examples, the computing device can include a mobile device, a wearable device, an XR device, a personal computer, a laptop computer, a video server, a video game console, a robotic device, a set-top box, a television, a camera, a server, or other device. For example, the computing device architecture 700 can implement the neural P-frame coding system 800 of FIG. 8. The components of computing device architecture 700 are shown in electrical communication with each other using connection 705, such as a bus. The example computing device architecture 700 includes a processing unit (CPU or processor) 710 and computing device connection 705 that couples various computing device components including computing device memory 715, such as read only memory (ROM) 720 and random access memory (RAM) 725, to processor 710.

Computing device architecture 700 can include a cache of high-speed memory connected directly with, in close proximity to, or integrated as part of processor 710. Computing device architecture 700 can copy data from memory 715 and/or the storage device 730 to cache 712 for quick access by processor 710. In this way, the cache can provide a performance boost that avoids processor 710 delays while waiting for data. These and other modules can control or be configured to control processor 710 to perform various actions. Other computing device memory 715 may be available for use as well. Memory 715 can include multiple different types of memory with different performance characteristics. Processor 710 can include any general purpose processor and a hardware or software service, such as service 1 732, service 2 734, and service 3 736 stored in storage device 730, configured to control processor 710 as well as a special-purpose processor where software instructions are incorporated into the processor design. Processor 710 may be a self-contained system, containing multiple cores or processors, a bus, memory controller, cache, etc. A multi-core processor may be symmetric or asymmetric.

To enable user interaction with the computing device architecture 700, input device 745 can represent any number of input mechanisms, such as a microphone for speech, a touch-sensitive screen for gesture or graphical input, keyboard, mouse, motion input, speech and so forth. Output device 735 can also be one or more of a number of output mechanisms known to those of skill in the art, such as a display, projector, television, speaker device, etc. In some instances, multimodal computing devices can enable a user to provide multiple types of input to communicate with computing device architecture 700. Communication interface 740 can generally govern and manage the user input and computing device output. There is no restriction on operating on any particular hardware arrangement and therefore the basic features here may easily be substituted for improved hardware or firmware arrangements as they are developed.

Storage device 730 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 725, read only memory (ROM) 720, and hybrids thereof. Storage device 730 can include services 732, 734, 736 for controlling processor 710. Other hardware or software modules are contemplated. Storage device 730 can be connected to the computing device connection 705. In one aspect, a hardware module that performs a particular function can include the software component stored in a computer-readable medium in connection with the necessary hardware components, such as processor 710, connection 705, output device 735, and so forth, to carry out the function.

The term “device” is not limited to one or a specific number of physical objects (such as one smartphone, one controller, one processing system, and so on). As used herein, a device can include any electronic device with one or more parts that may implement at least some portions of this disclosure. While the description and examples use the term “device” to describe various aspects of this disclosure, the term “device” is not limited to a specific configuration, type, or number of objects. Additionally, the term “system” is not limited to multiple components or specific examples. For example, a system may be implemented on one or more printed circuit boards or other substrates, and may have movable or static components. While the description and examples use the term “system” to describe various aspects of this disclosure, the term “system” is not limited to a specific configuration, type, or number of objects.

Specific details are provided in the description to provide a thorough understanding of the aspects and examples provided herein. However, it will be understood by one of ordinary skill in the art that the aspects may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the aspects in unnecessary detail. In other instances, well-known circuits, processes, algorithms, structures, and techniques may be shown without unnecessary detail in order to avoid obscuring the examples.

Individual aspects and/or examples may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as flash memory, memory or memory devices, magnetic or optical disks, flash memory, USB devices provided with non-volatile memory, networked storage devices, compact disk (CD) or digital versatile disk (DVD), any suitable combination thereof, among others. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some aspects, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific examples thereof, but those skilled in the art will recognize that the application is not limited thereto. Thus, while illustrative examples of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, aspects of the present disclosure can be utilized in any number of environments and applications beyond those described herein without departing from the scope of the specification. The specification and drawings are, accordingly, to be regarded as illustrative rather than restrictive. For the purposes of illustration, methods were described in a particular order. It should be appreciated that in alternate examples, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random-access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

What is claimed is:

1. One or more methods as described and disclosed in the accompanying Summary, Detailed Description and disclosures accompanying this application.

2. One or more apparatuses as described and disclosed in the accompanying Summary, Detailed Description and disclosures accompanying this application.
